(19) United States
(12) Patent Application Publication
(10) Pub. No.: US 2025/0185892 A1
(43) Pub. Date: Jun. 12, 2025

(54) MEDICAL DEVICE ACTUATORS

(71) Applicant: Boston Scientific Scimed, Inc., Maple Grove, MN (US)

(72) Inventors: Sean POWELL, Holden, MA (US); James WELDON, Newton, MA (US); Matthew Robert JAGELSKI, Milford, MA (US)

(73) Assignee: Boston Scientific Scimed, Inc., Maple Grove, MN (US)

(21) Appl. No.: 18/966,403

(22) Filed: Dec. 3, 2024

Related U.S. Application Data
(60) Provisional application No. 63/606,674, filed on Dec. 6, 2023.

(51) Int. Cl. A61B 1/00

(52) U.S. Cl. CPC A61B 1/00133

(57) ABSTRACT

An actuation assembly for a medical device may include a first gear including: a first gear toothed region; and a first gear untoothed region; and a second gear including: a second gear toothed region; a second gear untoothed region; and an articulation wire coupled to the second gear. In a first portion of a rotational range of the first gear, the first gear toothed region may interact with the second gear toothed region, such that the second gear toothed region rotates upon rotation of the first gear. In a second portion of the rotational range, the first gear untoothed region may interact with the second gear untoothed region, such that the second gear remains stationary upon rotation of the first gear.

### CROSS-REFERENCE TO RELATED APPLICATIONS

This application claims the benefit of priority to U.S. Provisional Application No. 63/606,674, filed on Dec. 6, 2023, which is incorporated by reference herein in its entirety.

### TECHNICAL FIELD

Various aspects of this disclosure relate generally to devices and methods for actuators of medical devices, and, in particular, to actuators that manipulate more than one wire to articulate a medical device in a given direction.

### BACKGROUND

Medical devices, such as scopes (e.g., endoscopes, duodenoscopes, etc.), may include a handle and a sheath/shaft insertable into a body lumen of a subject. The shaft may terminate in a distal tip portion, which may include features such as elevators, optical elements (e.g., camera, lighting, etc.), air/water outlets, working channel openings, and/or accessory devices. Actuators in the handle of the scope may control actuatable elements of the shaft and/or distal tip. For example, buttons, knobs, levers, etc. may control elements of the shaft and/or distal tip. Control elements (e.g., wires, cables, or shafts) may be utilized to transmit a force to a distal portion of the sheath. Such control elements may be coupled to control mechanisms in a handle, such as pulleys or levers. When a control mechanism is actuated by an actuator, a force may be transferred via a control element, thereby actuating elements of the shaft or distal tip. For example, a control mechanism may be coupled to an articulation joint of the medical device to allow for steering of a distal portion of the shaft and the distal tip. A need exists for improved actuation assemblies to provide control of distal portions of medical devices.

### SUMMARY

Each of the aspects disclosed herein may include one or more of the features described in connection with any of the other disclosed aspects.

Aspects of the disclosure relate to, among other things, systems, devices, and methods for actuators of medical devices. The medical device may include a shaft having more than one wire that controls articulation in a direction (e.g., up, down, left, or right). A single actuator (e.g., a knob) may control the more than one wire.

In an example, an actuation assembly for a medical device may include a first gear including: a first gear toothed region; and a first gear untoothed region; and a second gear including: a second gear toothed region; a second gear untoothed region; and an articulation wire coupled to the second gear. In a first portion of a rotational range of the first gear, the first gear toothed region may interact with the second gear toothed region, such that the second gear toothed region rotates upon rotation of the first gear. In a second portion of the rotational range, the first gear untoothed region may interact with the second gear untoothed region, such that the second gear remains stationary upon rotation of the first gear.

Any of the assemblies disclosed herein may include any of the following features, alone or in any combination. The articulation wire may be a first articulation wire. The actuation assembly may further comprise: a third gear configured to interact with the first gear; and a second articulation wire coupled to the third gear. The third gear may include: a third gear toothed region; and a third gear untoothed region. The first gear toothed region may be a first gear first toothed region. The first gear untoothed region may be a first gear first untoothed region. The articulation wire may be a first articulation wire. The first gear may have a first portion. The first portion may include the first gear first toothed region and the first gear first untoothed region. The first gear also may have a second portion. The second portion may include: a first gear second toothed region; and a first gear second untoothed region. The third gear may be configured to interact with the second portion of the first gear. The first portion may be on a first axial side of the first gear, wherein and the second portion is on a second axial side if the first gear. The second gear and the third gear may be coaxial. In the first portion of a rotational range of the first gear, the first gear may interact with the third gear such that the third gear remains stationary upon rotation of the first gear. The first articulation wire may have a distal end that is coupled to a first portion of an articulation joint. The second articulation wire may have a distal end that is coupled to a second portion of the articulation joint. The first portion of the articulation joint may be proximal of the second portion of the articulation joint. The first articulation wire and the second articulation wire may be coupled to the articulation joint at a same angular position of the articulation joint. The actuation assembly may further comprise a knob. Rotation of the knob may be configured to control movement of both the first articulation wire and the second articulation wire. The second gear untoothed region may be concave. The second gear untoothed region may be a second gear first untoothed region. The second gear may further include a second gear second untoothed region. The first gear or the second gear may be non-circular. The third gear may benon-circular.

In another example, an actuation assembly for a medical device may comprise: a knob; a first member coupled to a first pair of articulation wires; and a second member coupled to a second pair of articulation wires. In a first portion of a rotational range of the knob, rotation of the knob may cause movement of the first pair of articulation wires while the second pair of articulation wires remain stationary. In a second portion of the rotational range of the knob, rotation of the knob may cause movement of the second pair of articulation wires.

Any of the assemblies disclosed herein may have any of the following features, alone or in any combination. The knob may be coupled to a first gear. The first member may be a second gear. The second member may be a third gear. The first gear includes a toothed region and an untoothed region. An actuation assembly for a medical device may comprise: a first gear; a second gear; and a third gear. In a first portion of a rotation range of the first gear, teeth of the first gear may engage teeth of the second gear to rotate the second gear; and an untoothed portion of the first gear engages an untoothed portion of the third gear, such that the third gear remains stationary. In a second portion of a rotation range of the first gear, teeth of the first gear may engage teeth of the third gear to rotate the third gear. The second gear may be coupled to a first articulation wire. The third gear may be coupled to a second articulation wire.

The aspects discussed above may be combined in any suitable combination or sub-combination.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 depicts an exemplary medical device.

FIG. 2 is an exploded view of an exemplary actuation assembly for use with the medical device of FIG. 1.

FIGS. 3A-3C depict an exemplary first gear of the actuation assembly of FIG. 2.

FIG. 4A is partially-transparent a side view of the actuation assembly of FIG. 2.

FIGS. 4B and 4C are cross-sectional views of the actuation assembly depicted in FIG. 4A.

FIGS. 5A-5D depict an exemplary second gear of the actuation assembly of FIGS. 2 and 4A-4C.

FIGS. 6A-6D depict an exemplary third gear of the actuation assembly of FIGS. 2 and 4A-4C.

FIG. 7 depicts an exemplary articulation joint of the medical device of FIG. 1.

FIGS. 8A-11B depict exemplary configurations of the articulation joint of FIG. 7 and corresponding configurations of the actuation assembly of FIGS. 2 and 4A-4C.

FIG. 12 depicts exemplary alternative gears for use with the actuation assembly of FIGS. 2 and 4A-4C.

FIGS. 13 and 14 depict exemplary alternative articulation joints.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator/toward a treatment site, and the term “proximal” refers to a direction toward an operator. One or more of the drawings include arrows labeled “P” and “D,” referring to proximal and distal directions, respectively. The term “exemplary” is used in the sense of “example,” rather than “ideal.” The term “approximately,” or like terms (e.g., “substantially”), includes values+/−10% of a stated value.

A medical device, such as a duodenoscope or endoscope, may include a shaft configured to be inserted into a body lumen of a subject and a handle configured to control aspects of the shaft. A distal portion of the shaft may include an articulation joint that may facilitate steering of the distal portion of the shaft. The articulation joint may have a plurality of control members (e.g., wires or cables) coupled to portions of the articulation joint. To accommodate the limited space available inside of a body lumen of a subject, it may be desirable to articulate a portion of an articulation joint (e.g., a distal portion of an articulation joint) to a tightly curled configuration before articulating another portion (e.g., a proximal portion of the articulation joint).

In some examples, to provide for articulation of one portion of the articulation joint before another portion of the articulation joint, the medical device may have more than one control wire for controlling articulation of the shaft in a single direction. For example, the articulation joint may have distal ends of two control members coupled to the articulation joint for controlling one of up, down, left, or right movement of the shaft.

In aspects of this disclosure, a single knob may be used to control the articulation of both control members associated with a given direction of articulation. In an example, a single knob of a steering assembly may be used to control up/down articulation of an articulation joint, and up/down articulation may be controlled by four control members (e.g., two for upward articulation and two for downward articulation). The knob may be coupled to a shaft, which may cause rotation of a first gear, which may be a driving gear. The driving gear may optionally be a pinion gear. The second gear may have two sides (e.g., a proximal side and a distal side), and each side may include a toothed region and an untoothed region. The steering assembly may also include a second gear, which may be coupled to two control members, and a third gear, which may similarly be coupled to two other control members. Each of the second gear and the third gear may include toothed regions and untoothed regions. As the firsts gear rotates, the toothed and untoothed regions of the first gear may selectively interact with the toothed and untoothed regions of the second and third gears, such that the second and third gears rotate or do not rotate, depending on which portions of the gear are interacting. Thus, throughout a range of rotation of the knob, control members coupled to the second gear and the third gear (which may be coupled to the articulation joint) may be tensioned or untensioned (i.e., de-tensioned) depending upon an interaction of the toothed and untoothed regions of the gears.

Although the term duodenoscope may be used herein, it will be appreciated that the disclosure encompasses actuation mechanisms for other devices, including, but not limited to, endoscopes, colonoscopes, ureteroscopes, bronchoscopes, laparoscopes, cytoscopes, hysteroscopes, sheaths, catheters, any other suitable delivery device, an accessory device for use with a delivery device (such as a scope), or another type of medical device including an actuator. Additionally, although side-facing devices may be referenced, the embodiments described herein may also be used with front-facing devices (e.g., devices where a viewing element faces longitudinally forward) or with devices that are a combination of side-facing and forward-facing. Furthermore, although the assemblies described below are discussed with respect to steering/articulating a shaft of the medical device, it will be appreciated that the assemblies may be used for alternative types of actuation.

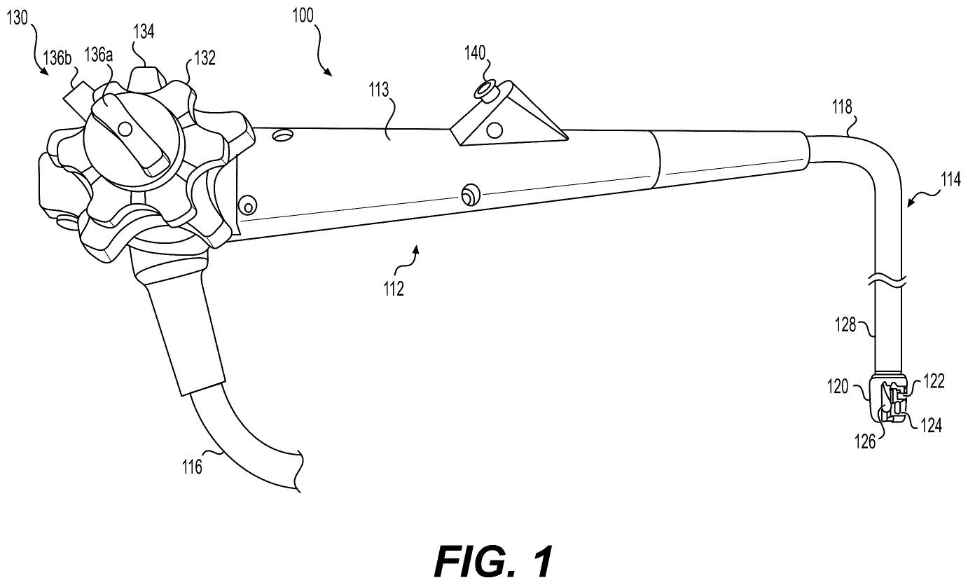

FIG. 1 depicts an exemplary medical device 100 (e.g., a duodenoscope) having a handle 112 and an insertion portion 114. Medical device 100 may also include an umbilicus 116 for purposes of connecting medical device 100 to sources of, for example, air, water, suction, power, etc., as well as to image processing and/or viewing equipment.

Insertion portion 114 may include a sheath or shaft 118 and a distal tip 120. Distal tip 120 may include an imaging device 122 (e.g., a camera) and a lighting source 124 (e.g., an LED or an optical fiber). Distal tip 120 may be side-facing. That is, imaging device 122 and lighting source 124 may face radially outward, perpendicularly, approximately perpendicularly, or otherwise transverse to a longitudinal axis of shaft 118 and distal tip 120. Alternatively, distal tip 120 may be forward-facing (imaging device 122 and/or lighting source 124 may face distally).

Distal tip 120 may also include an actuatable element, for example an elevator 126 for changing an orientation of a tool inserted in a working channel of medical device 100. Elevator 126 may alternatively be referred to as a swing stand, pivot stand, raising base, or any suitable other term. Elevator 126 may be pivotable via, for example, an actuation wire or another control element that extends from handle 112, through shaft 118, to elevator 126.

A distal portion of shaft 118 that is connected to distal tip 120 may have a steerable section 128. Steerable section 128 may include, for example, an articulation joint, such as the articulation joints discussed below. Shaft 118 and steerable section 128 may include a variety of structures which are known or may become known in the art. In some examples, one or more steering wires may be coupled to steerable section 128 so that, as the steering wires are tensioned/de-tensioned, steerable section 128 articulates in one or more directions.

Handle 112 may have a housing 113 that encases/houses various elements of handle 112. Handle 112 may have one or more actuators/control mechanisms 130. Control mechanisms 130 may provide control over steerable section 128 or may allow for provision of air, water, suction, etc. For example, handle 112 may include a first control knob 132 and a second control knob 134 for left, right, up, and/or down control of steerable section 128. For example, first knob 132 may provide left/right control of steerable section 128, and second knob 134 may provide up/down control of steerable section 128. Handle 112 may further include one or more locking mechanisms 136a, 136b (e.g., knobs or levers) for preventing steering of steerable section 128 in at least one of an up, down, left, or right direction. Handle 112 may include an elevator control mechanism, which is not shown in FIG. 1. A port 140 may allow passage of a tool through port 140, into a working channel of the medical device 100, through shaft 118, to distal tip 120.

In use, an operator may insert at least a portion of shaft 118 into a body lumen of a subject. Distal tip 120 may be navigated to a procedure site in the body lumen. The operator may use knobs 132, 134 to steer steerable section 128 of shaft 118 to a desired position. The operator may insert an accessory device, such as an instrument (not shown) into port 140, and pass the tool through shaft 118 via a working channel to distal tip 120. The tool may exit the working channel at distal tip 120. The operator may use an elevator control mechanism (not shown) to raise elevator 126 and angle the accessory device toward a desired location (e.g., a papilla of the pancreatico-biliary tract). The operator may use the accessory device to perform a medical procedure.

Although FIG. 1 depicts a side-viewing duodenoscope, it will be appreciated that, as discussed above, this disclosure is not so limited. The aspects discussed herein may alternatively be utilized with other types of medical devices. For example, the embodiments disclosed herein may be utilized with any type of scope.

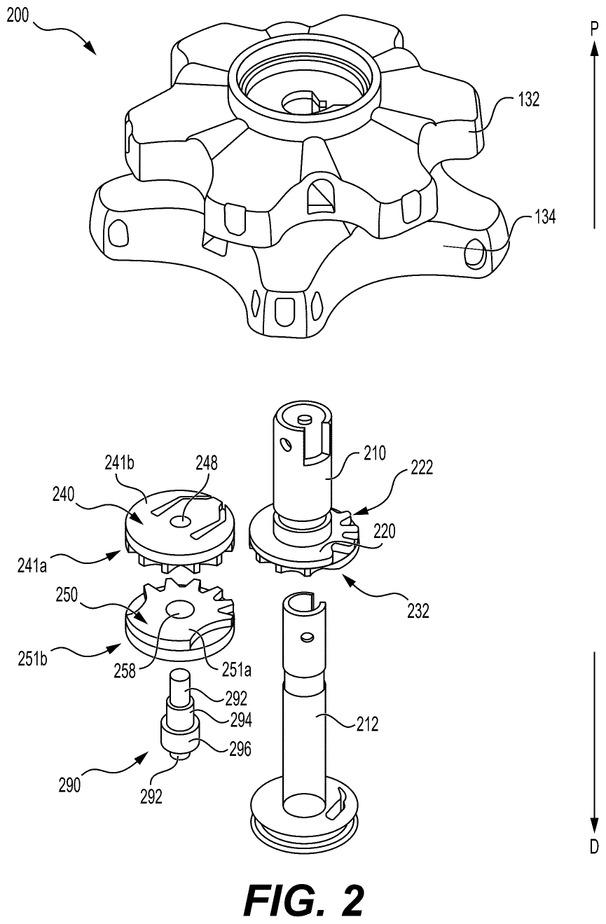

FIG. 2 depicts an exploded view of a steering assembly 200 of medical device 100. First knob 132 and second knob 134 may be external to housing 113 of handle 112. In some examples, first knob 132 may be a left/right steering knob, and second knob 134 may be an up/down steering knob. In other examples, first knob 132 may be an up/down steering knob, and second knob 134 may be a left/right steering knob. Alternatively, knobs 132 and 134 (and other elements of steering assembly 200 described herein) may actuate other elements of medical device 100 or may articulate an articulation joint of steerable section 128 of shaft 118 in other directions.

Other elements of steering assembly 200 may be at least partially within an interior of housing 113 of handle 112. Second knob 134 may be coupled to (e.g., fixedly coupled to) a shaft 210. In some examples, shaft 210 may define a central lumen, through which another shaft 212 may be received. As used herein, the term “axial” may refer to a direction coaxial with or parallel to a central longitudinal axis of shaft 210. In aspects, shaft 212 may be coupled to (e.g., fixedly coupled to) first knob 132. Although functionality of first knob 132 and shaft 212 are not described herein, it will be appreciated that first knob 132 may be used to articulate a variety of wires for steering or for actuation of various aspects of distal tip 120 of medical device 100. First knob 132 and shaft 212 may be coupled to similar structures to the structures of steering assembly 200 associated with second knob 134 and described herein, or may be coupled to other types of actuation mechanisms or assemblies (e.g., pulleys, linkages, etc.).

Shaft 210 may be coupled to (e.g., fixedly coupled to) a first gear 220 or other member, such as a driving, pinion, spur, or other type of gear. First gear 220 may interact with a second gear 240 or other member and a third gear 250 or other member, described in further detail below. Second gear 240 and third gear 250 may each be coupled to one or more articulation wires for articulating steerable section 128. The terms “first,” “second,” and “third” are used herein for convenience of reference and do not imply any ordinal relationship. Any of the gears described herein may be “first,” “second,” or “third” gears.

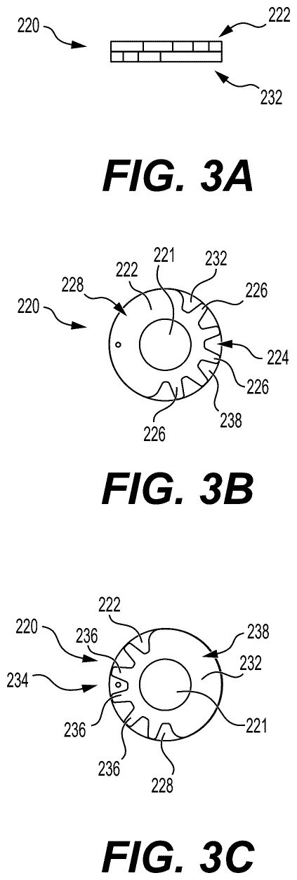

First gear 220 may have a cylindrical or plate shape and may have approximately circular proximal and distal faces. FIGS. 3A-3C show first gear 220 in further detail. First gear 220 may have a first portion 222 and a second portion 232. First portion 222 may be on a first axial side of first gear 220 and may define a first face of first gear 220. Second portion 232 may be on a second axial side (opposite to the first axial side) of first gear 220 and may define a second face of first gear 220. First gear 220 may have an approximately annular shape, with a central opening 221 for receiving shaft 210. In alternatives, first portion 222 and second portion 232 may be separate pieces (separate gears) that are stacked on top of one another on a common axle. In further examples, multiple first gears 220 may be used, each having one or more portions.

As shown in FIG. 3B, first portion 222 of first gear 220 may include a toothed region 224 having a plurality of teeth 226 (e.g., gear teeth). First portion 222 may also include an untoothed (e.g., smooth) region 228 (a first gear first untoothed region). Untoothed region 228 may have an approximately arcuate, convex shape (e.g., a portion of a circumference of first gear 220). A circular shape may be drawn around an outer edge of untoothed region 228 and along outer surfaces of teeth 226. As shown in FIG. 3B, toothed region 224 may be approximately half of a perimeter/circumference of first portion 222, and untoothed region 228 may be approximately half of the perimeter/circumference of first portion 222. However, such proportions are not limiting, and, depending on a desired function of steering assembly 200, may differ from the arrangement shown in FIG. 3B.

As shown in FIG. 3C, second portion 232 of first gear 220 may be similar to first portion 222. Second portion 232 of first gear 220 may include a toothed region 234 (a first gear second untoothed region) having a plurality of teeth 236 (e.g., gear teeth). Second portion 232 may also include an untoothed (e.g., smooth) region 238 (a first gear second untoothed region). Untoothed region 238 may have an approximately arcuate shape (e.g., a portion of a circumference of first gear 220). A circular shape may be drawn around an outer edge of untoothed region 238 and along outer surfaces of teeth 236. As shown in FIG. 3B, toothed region 234 may be approximately half of a perimeter/circumference of second portion 232, and untoothed region 238 may be approximately half of the perimeter/circumference of second portion 232. However, such proportions are not limiting, and depend on a desired function of steering assembly 200.

As shown in FIGS. 3A and 3B, second portion 232 (e.g., untoothed region 238) may be visible between teeth 226 of first portion 222 when viewing first portion 222, as in FIG. 3B. Similarly, when viewing second portion 232 as in FIG. 3C, first portion 222 (e.g., untoothed region 228) may be visible between teeth 236 of second portion 232.

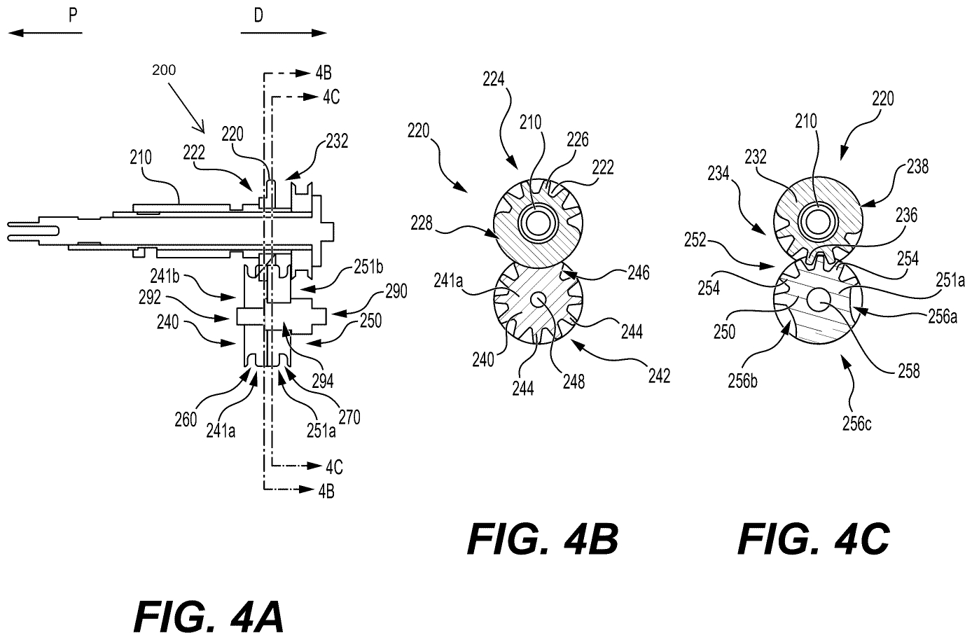

FIG. 4A shows a partially transparent side view of a portion of steering assembly 200. FIG. 4B shows a cross-section of the view of FIG. 4A taken along line 4B. FIG. 4C shows a cross-section of the view of FIG. 4A taken along line 4C. As shown in FIGS. 4A-4C, first gear 220 may interact with a second gear 240 and a third gear 250. Second gear 240 and third gear 250 may be approximately cylindrical or approximately plate shaped and may have approximately circular faces. In examples, second gear 240 and third gear 250 may be spur gears. As shown in FIG. 4A, second gear 240 and third gear 250 may be stacked axially, such that second gear 240 interacts with first portion 222 of first gear 220, and third gear 250 interacts with second portion 232 of first gear 220. In examples, a boundary between second gear 240 and third gear 250 may be aligned axially with a boundary between first portion 222 and second portion 232.

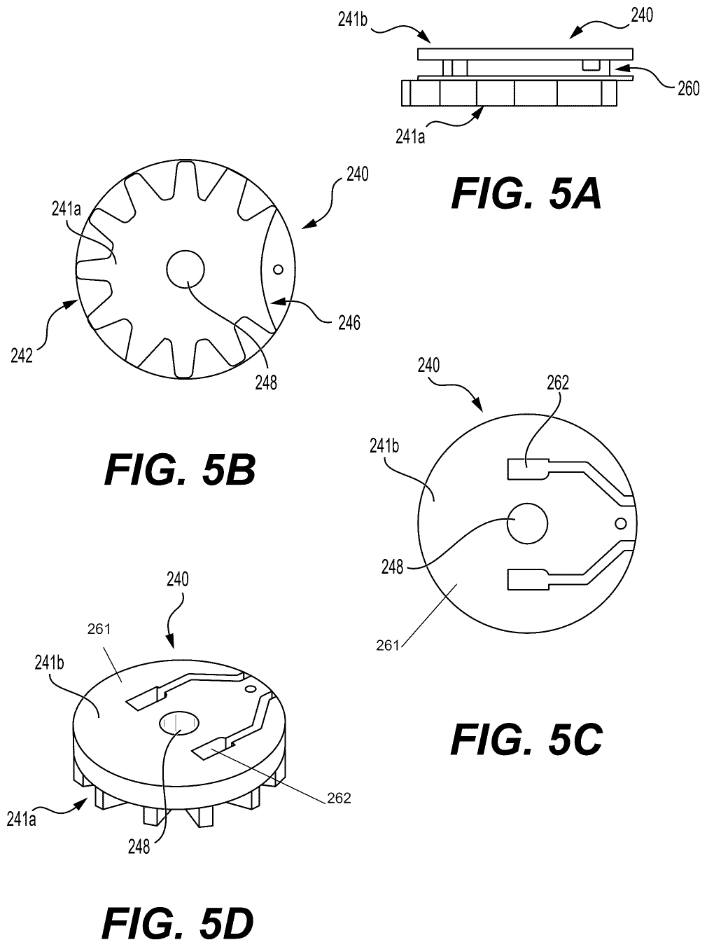

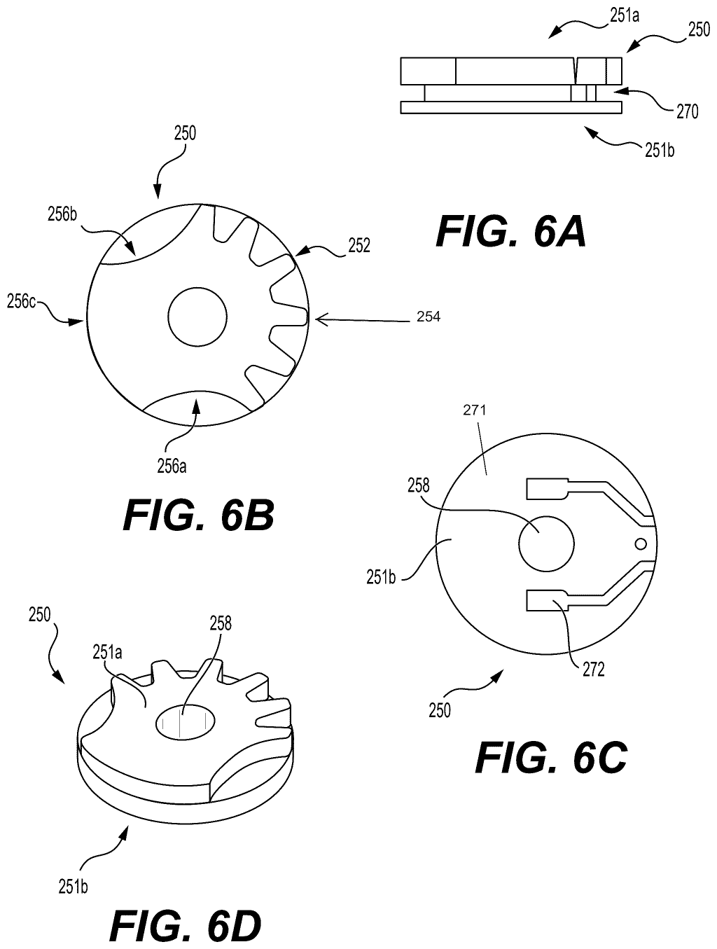

FIGS. 5A-6D show various views of second gear 240 and third gear 250. As shown in FIG. 5A, second gear 240 may have a first portion 241a on a first axial side of second gear 240 and a second portion 241b on a second axial side of second gear 240. As shown in FIGS. 4B and 5A, first portion 241a of second gear 240 may include a toothed region 242 (a second gear toothed region) having a plurality of teeth 244 and an untoothed region 246 (a second gear untoothed region). Untoothed region 246 may have a concave shape. A curve drawn along outer surfaces of teeth 244 may form an arc that is a portion of a circle. However, because untoothed region 246 is concave, first portion 241a may have an outer perimeter that is only partially circular (with the circular portion corresponding to toothed region 242). At least portions of untoothed region 246 may be recessed relative to a circular curve extending along outer surfaces of teeth 244. Portions of untoothed region 246 may also be recessed relative to a circular curve extending around radially inner sides of teeth 244. Relative sizes of untoothed region 246 and toothed region 242 may be chosen based on a desired functionality of steering assembly 200.

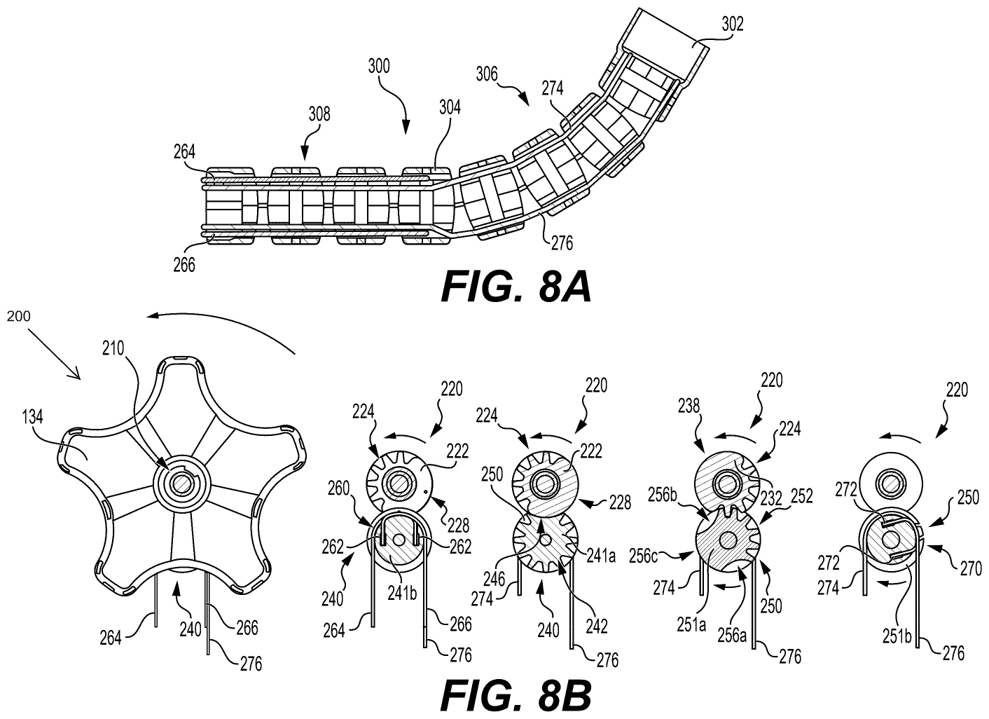

As shown in FIG. 5A, second portion 241b may include a groove or other recess 260 Recess 260 may be between a face 261 of second portion 241b and first portion 241a. Recess 260 may receive one or more control members, as shown in FIG. 8B and described below. The control members may be wrapped around second portion 241b within recess 260, such that second portion 241b may be a pulley for tensioning and de-tensioning (e.g., lengthening and shortening) control members wrapped around second portion 241b. As shown in FIG. 50, a face 261 of second portion 241b may have one or more openings 262 formed thereon (e.g., two openings 262). Each of openings 262 may receive ends of one or more control members to secure the control member(s) to second portion 241b/recess 260.

As shown in FIG. 6A third gear 250 may have a first portion 251a on a first axial side of third gear 250 and a second portion 251b on a second axial side of third gear 250. As shown in FIGS. 4C and 6B, third gear 250 may include a toothed region 252 (a third gear toothed region) having a plurality of teeth 254. Third gear 250 may also have a first untoothed region 256a (a third gear first untoothed region), a second untoothed region 256b (a third gear second untoothed region), and a third untoothed region 256c (a third gear third untoothed region). Untoothed regions 256a, 256b may have concave shapes, similar to untoothed region 246. Untoothed regions 256a, 256b may be recessed relative to teeth 254, as discussed above for second gear 240. Third untoothed region 256c may have a convex shape. A circle drawn so that it contacts outer surfaces of teeth 254 may also contact an outer surface of third untoothed region 256c. Third untoothed region 256c may be between first and second untoothed regions 256a, 256b (e.g., directly adjacent to first and second untoothed regions 256a, 256b). Third untoothed region 256c may be approximately diametrically opposed to toothed portion 252 (i.e., may be on opposite ends of a line drawn through a center of third gear 250 perpendicularly to the axial direction). In examples, each of untoothed regions 256a and 256b may extend around approximately 30 degrees of third gear 250. Third untoothed region 256c may extend around approximately 60 degrees of third gear 250. Toothed region 252 may extend around approximately 120 degrees of third gear 250.

Second portion 251b may have any of the same properties as second portion 241b. Second portion 251b may include a groove or other recess 270. Recess 270 may receive one or more control members, as shown in FIG. 8B and described below. The control members may be wrapped around second portion 251b within recess 270, such that second portion 251b may be a pulley for tensioning and detensioning (e.g., lengthening and shortening) control members wrapped around second portion 251b. As shown in FIG. 5C, a face 271 of second portion 251b may have one or more openings 272 formed thereon (e.g., two openings 272). Each of openings 272 may receive ends of one or more control members to secure the control member(s) to second portion 251b/recess 270.

As shown in FIGS. 5B-5D, an opening 248 may extend through an entire thickness of second gear 240, through first portion 241a and second portion 241b, Opening 248 may be at a center of second gear 240. Additionally, as shown in FIGS. 6B-6D, an opening 258 may extend through an entire thickness of third gear 250, through first portion 251a and second portion 251b. Opening 258 may be at a center of third gear 250. Openings 248 and 258 may have a circular cross-sectional shape or any other suitable shape. In some examples, opening 248 may have a smaller diameter than opening 258. Alternative, opening 248 may have a larger diameter than opening 258 or may have an equal diameter to opening 258.

Referring to FIGS. 2 and 4A, second gear 240 and third gear 250 may be arranged such that first portion 241a of second gear 240 and first portion 251a of third gear 250 face one another and are axially adjacent to one another. As shown, Second gear 240 may be proximal of third gear 250. In alternatives, third gear 250 may be proximal of second gear 240. Second portion 241b may be proximal of first portion 241a, which may be proximal of first portion 251a, which may in turn be proximal of second portion 251b.

A retention mechanism 290 (e.g., a shaft, a pin, etc.) may extend through opening 248 of second gear 240 and opening 258 of third gear 250. Retention mechanism 290 may form an axle. Second gear 240 and third gear 250 may be rotatable with respect to retention mechanism 290, such that a center of retention mechanism 290 forms a central axis of second gear 240 and third gear 250, about which second gear 240 and third gear 250 may rotate. As shown in FIG. 4A, a central longitudinal axis of retention mechanism 290 may be substantially parallel to a central longitudinal axis of shaft 210. The central longitudinal axis of retention mechanism 290 may be offset (not coaxial with) the central longitudinal axis of shaft 210.

As shown in FIG. 2, retention mechanism 290 may have a non-uniform width/diameter along a longitudinal length of retention mechanism 290. For example, retention mechanism 290 may have an inner shaft portion 292. Inner shaft portion 292 may be exposed at proximal and distal ends of retention mechanism 290. Retention mechanism 290 may have an intermediate shaft portion 294, which may be disposed around a portion of retention mechanism 290, between proximal and distal ends of retention mechanism 290. Retention mechanism 290 may have an outer shaft portion 296, which may be disposed around a portion of intermediate shaft portion 294. Inner shaft portion 292, intermediate shaft portion 294, and outer shaft portion 296 may be separate elements that are fixedly coupled to one another or may integrally formed from a single, monolithic piece of material.

Inner shaft portion 292 may have a width/diameter that is slightly smaller than a diameter of opening 248, such that a proximal end of retention mechanism 290 (e.g., inner shaft portion 292) may be received within opening 248 and second gear 240 may rotate relative to retention mechanism 290. Intermediate shaft portion 294 may have a width/diameter that is larger than a diameter of opening 248 such that intermediate portion 294 remains distal to second gear 240. Intermediate shaft portion 294 may have a width/diameter that is slightly smaller than a diameter of opening 258, such that intermediate shaft portion 294 may be received within opening 258 and third gear 250 may rotate with respect to retention mechanism 290. Outer shaft portion 296 may have a width/diameter that is larger than a diameter of opening 258, such that outer shaft portion 296 remains distal of third gear 250. Retention mechanism 290 may help to secure second gear 240 and third gear 250 in a desired position within steering assembly 200 and/or may facilitate rotation of second gear 240 and third gear 250 about their central longitudinal axes.

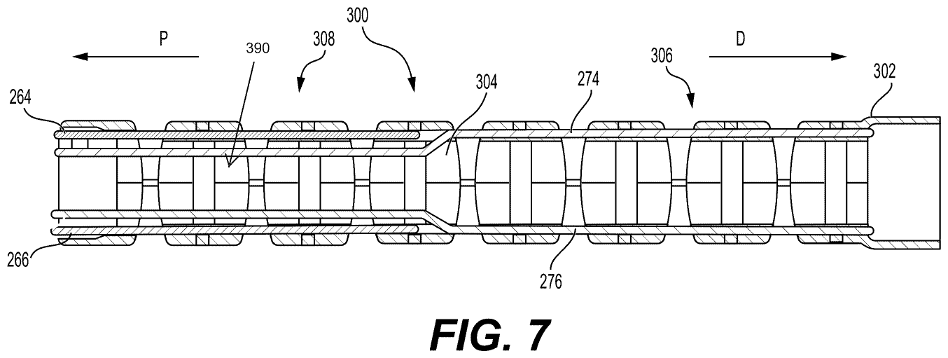

FIG. 7 depicts an exemplary articulation joint 300 of shaft 118 of medical device 100. As shown in FIG. 7, a first articulation wire 264 and a second articulation wire 266 may be coupled (e.g., fixedly coupled) to an intermediate link 304 of articulation joint 300. First articulation wire 264 and second articulation wire 266 may be a pair of articulation wires and may be diametrically opposite to one another and coupled to the same proximal structure (e.g., second gear 240). Intermediate link 304 may be disposed between a proximal end and a distal end of articulation joint 300 (e.g., approximately midway between the proximal end and the distal end of articulation joint 300). First articulation wire 264 and/or second articulation wire 266 may be movably coupled to links of articulation joint 300 that are in a proximal portion 308 of articulation joint 300, proximal of link 304. First articulation wire 264 and second articulation wire 266 may be coupled to opposite sides of link 304 (e.g., diametrically opposed to one another). In examples, articulation wire 264 may be associated with upward movement of articulation joint 300, and articulation wire 266 may be associated with downward movement of articulation joint 300. However, such directions are merely exemplary.

It will be appreciated that wires 264, 266, 274, and 276 may have different intermediate links to which they are coupled. For example, distal ends of wires 264 and 266 may be coupled to different links of articulation joint 300. In other words, one of wires 264 or 266 may terminate distally of the other of wires 264 or 266. Similarly, a proximalmost link of articulation joint 300 to which wires 274 and 276 are coupled may vary between wires 274 and 276. In other words, one of wires 274, 276 may be coupled to articulation joint 300 at a point that is proximal of a proximalmost location where the other of wires 274, 276 is coupled to articulation joint 300. Proximalmost link(s) to which wires 274, 276 are coupled may vary from link(s) to which distal ends of wires 264, 266 are coupled.

A third articulation wire 274 and a fourth articulation wire 276 may be coupled (e.g., fixedly coupled) to a distal link 302 (e.g., a distalmost link) of articulation joint 300. Third articulation wire 274 and fourth articulation wire 276 may be a second pair of articulation wires. Third articulation wire 274 and fourth articulation wire 276 may be coupled to opposite sides of distal link 302 (e.g., diametrically opposed to one another). Third articulation wire 274 and fourth articulation wire 276 may be movably coupled to links of articulation joint 300 between link 304 and distal link 302. In other words, third articulation wire 274 and fourth articulation wire 276 may be movably coupled to links of a distal portion 306 of articulation joint 300. First articulation wire 264 and third articulation wire 274 may be coupled to the same angular position of articulation joint 300 (i.e., on a same side of articulation joint 300, at corresponding points of a circumference of the respective portions of articulation joint 300 to which first articulation wire 264 and third articulation wire 274 are coupled). Similarly, second articulation wire 266 and fourth articulation wire 276 may be coupled to the same angular position of articulation joint 300. Proximally of link 304, third articulation wire 274 and fourth articulation wire 276 may extend through a central lumen 390 of articulation joint 300. In examples, third articulation wire 274 may be associated with upward movement of articulation joint 300, and fourth articulation wire 276 may be associated with downward movement of articulation joint 300. However, such directions are merely exemplary.

In the configuration of FIG. 7, articulation joint 300 may have a neutral configuration. In other words, first articulation wire 264, second articulation wire 266 third articulation wire 274, and fourth articulation wire 276 may be unactuated, such that they have a relaxed configuration in which they have not been moved proximally or distally relative to one another.

FIGS. 8A-11B show steering assembly 200 in exemplary configurations, along with a corresponding configuration of articulation joint 300 at a distal portion of shaft 118 (FIG. 1). The configurations of FIGS. 8A-11B depict different portions of a range of rotation (i.e., a rotational range) of second knob 134 and first gear 220. The configurations shown in FIGS. 8A-11B and described below are merely exemplary, and steering assembly 200 may have any number of suitable configurations. FIGS. 8B, 9B, 10B, and 11B show knob 134 on the left and various cross-sectional views of steering assembly 200 in the four views on the right. Progressing in a rightward direction, the cross-sections may be increasingly distal cross-sections. In the rightmost cross-section of FIGS. 8B, 9B, 10B, and 11B, first gear 220 is not visible because the cross-section is taken distally of first gear 220. In FIG. 8B, articulation wires associated with third gear 250 are, in some views, visible behind second gear 240

As shown in FIG. 8B, second gear 240 may be coupled to first articulation wire 264 and second articulation wire 266. For example, first articulation wire 264 and second articulation wire 266 may be received within openings 262 of second gear 240 and may be wrapped around recess 260 (FIGS. 5A, 5C, and 5D). Third gear 250 may be coupled to third articulation wire 274 and fourth articulation wire 276. Third articulation wire 274 and fourth articulation wire 276 may be received within openings 272 of third gear 250 and may be wrapped around other recess 270 (FIGS. 6A, 6C, and 6D). Although articulation wires 264, 266, 274, 276 are not shown in the other Figures, it will be appreciated that they are similarly coupled to second gear 240 and third gear 250, as described above and shown in FIG. 8B.

In a first portion of the rotational range of knob 134 and first gear 220, knob 134 may be rotated counterclockwise in the view of FIG. 8B which may correspond to an “up” direction. It will be appreciated that the “up” direction is merely exemplary, and such rotation of knob 134 may correspond to any suitable direction. First gear 220 may rotate counterclockwise along with knob 134. Untoothed region 228 of first portion 222 of first gear 220 may engage/slide along untoothed region 246 of first portion 241a of second gear 240. Because untoothed region 228 is concave and lacks teeth, second gear 240 may thus not rotate as untoothed region 228 slides along untoothed region 246. Second gear 240 (along first articulation wire 264 and second articulation wire 266) may remain stationary. Simultaneously, toothed region 234 of second portion 232 of first gear 220 may interact with and engage with toothed region 252 of first portion 251a of third gear 250. This interaction between toothed region 234 and toothed region 252 may cause third gear 250 to rotate in a clockwise direction. As third gear 250 rotates, it may tension third articulation wire 274 (move third articulation wire 274 proximally) and de-tension fourth articulation wire 276 (move fourth articulation wire 276 distally).

As shown in FIG. 8A, the interactions of first gear 220, second gear 240, and third gear 250 above may result in third articulation wire 274 pulling proximally on an upper portion of distal link 302 and distal portion 306, causing distal portion 306 (a portion distal to link 304) to articulate upwardly. Because first articulation wire 264 and second articulation wire 266 remain in a relaxed configuration (are not tensioned) due to a lack of rotation of second gear 240, first articulation wire 264 and second articulation wire 266 may inhibit proximal portion 308 (a portion proximal of link 304) of articulation joint 300 from articulating. Thus, articulation joint 300 may form a first articulation shape, which may be a J-shape (e.g., an upwardly curving J shape). Proximal portion 308 may be substantially straight and distal portion 306 may be curved upward.

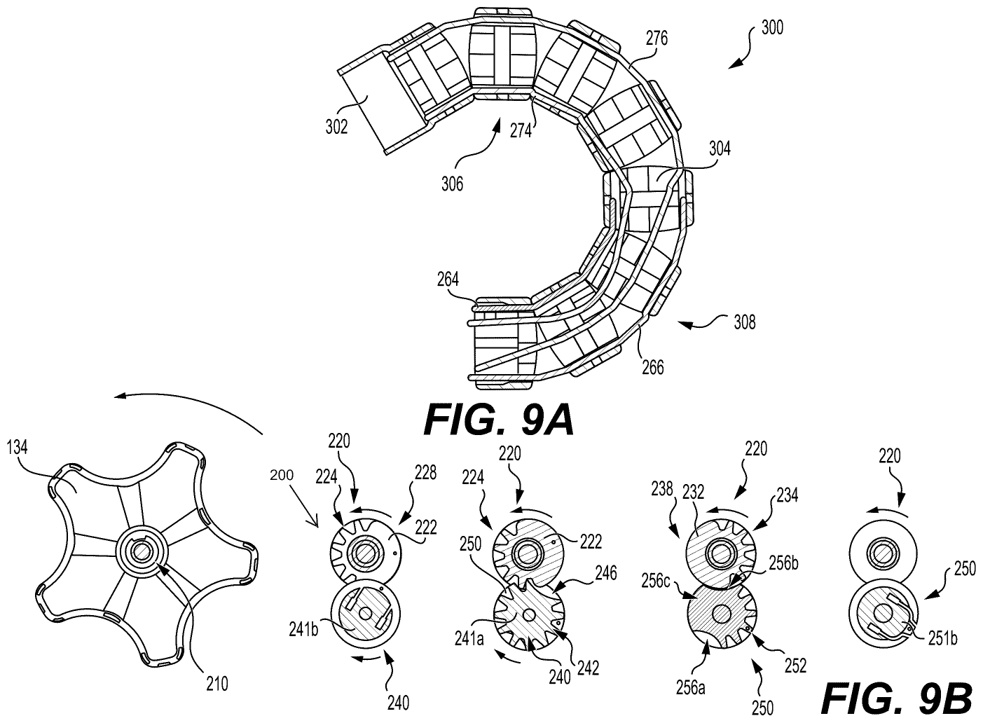

As shown in FIG. 9B, as second knob 134 continues to rotate in the counterclockwise direction, through a second portion of the rotational range, after a predetermined amount of rotation, untoothed region 238 of second portion 232 of first gear 220 engages/glides along second untoothed region 256b of 251a of third gear 250. Thus, third gear 250 stops rotating and remains stationary (along with third articulation wire 274 and fourth articulation wire 276). Third articulation wire 274 remains in tension as in the configuration of FIG. 8B. Meanwhile, rotation of first gear 220 is such that toothed region 224 of first portion 222 of first gear 220 engages toothed region 242 of first portion 241a of second gear 240. Thus, after a predetermined amount of rotation, second gear 240 begins to rotate in a clockwise direction of FIG. 9B. In some examples, second gear 240 may begin rotating at approximately the same time as third gear 250 ceases rotating. Alternatively, second gear 240 and third gear 250 may have overlapping rotational ranges. Rotation of second gear 240 applies tension to first articulation wire 264 (pulls first articulation wire 264 proximally) and de-tensions second articulation wire 266 (moves second articulation wire 266 distally). Second knob 134 may reach a stop (e.g., a hard stop), which prevents further counterclockwise rotation of second knob 134, along with first gear 220.

Thus, as shown in FIG. 9B, second articulation wire 266 applies a force on link 304 and proximal portion 308, causing proximal portion 308 to articulate upward. Distal portion 306 may remain in its articulated state. Thus, articulation joint 300 may adopt an upwardly curled shape. Articulation joint 300 may have second articulated shape, such as a tight curve (e.g., a “C” shape), as shown in FIG. 9A. Both distal portion 306 and proximal portion 308 may be curved in the second articulated configuration.

To straighten articulation joint 300 (to the configuration of FIG. 7), the reverse of the above steps may be performed. Second knob 134 may be rotated in a clockwise direction, thereby rotating first gear 220 in a clockwise direction. Initially, toothed region 224 of first portion 222 of first gear 220 may engage toothed region 242 of 240, thereby rotating second gear 240 counterclockwise and returning first articulation wire 264 and second articulation wire 266 to their relaxed states. Simultaneously, untoothed region 228 of first portion 222 of first gear 220 may engage second untoothed region 256b of 251a of third gear 250, such that third gear 250 does not rotate (remains stationary, along with third articulation wire 274 and fourth articulation wire 276). This action may transition articulation joint 300 to the shape of FIG. 8A.

Further clockwise rotation of second knob 134 may cause toothed region 224 of first portion 222 of first gear 220 to cease engaging toothed region 242 of second gear 240. Untoothed region 228 of first portion 222 may instead engage/slide by untoothed region 246 of second gear 240, such that second gear 240 does not rotate. Simultaneously, toothed region 234 of first gear 220 may engage toothed region 252 of third gear 250, thereby transitioning third articulation wire 274 and fourth articulation wire 276 to their neutral configurations and straightening articulation joint 300.

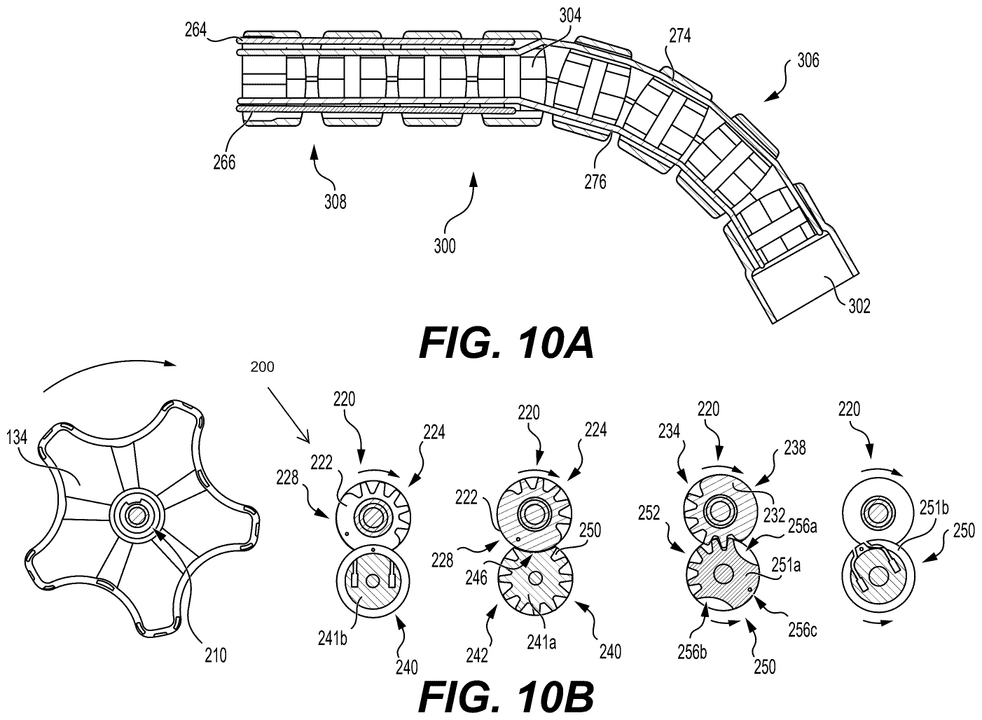

As shown in FIGS. 10A and 10B, when second knob 134 is rotated clockwise from its neutral position, through a portion of the rotational range of second knob 134 and first gear 220, first gear 220 may move clockwise along with second knob 134. Initially, toothed region 234 of second portion 232 of first gear 220 may engage toothed region 252 of third gear 250, thereby rotating third gear 250 counterclockwise. This may apply tension to fourth articulation wire 276 (move fourth articulation wire 276 proximally), articulating distal portion 306 of articulation joint 300 downward. Meanwhile, untoothed region 228 may engage (e.g., slide by) untoothed region 246 of second gear 240, such that second gear 240 may not rotate and remains stationary, along with first articulation wire 264 and second articulation wire 266. As explained below, the neutral state of first articulation wire 264 and second articulation wire 266 may inhibit proximal portion 308 of articulation joint 300 from articulating. Articulation joint 300 may have a third articulated configuration, which may be similar to the first articulated configuration and may have a J-shape (e.g., a downwardly bending J shape), as shown in FIG. 10A. Proximal portion 308 may be substantially straight, and distal portion 306 may be curved.

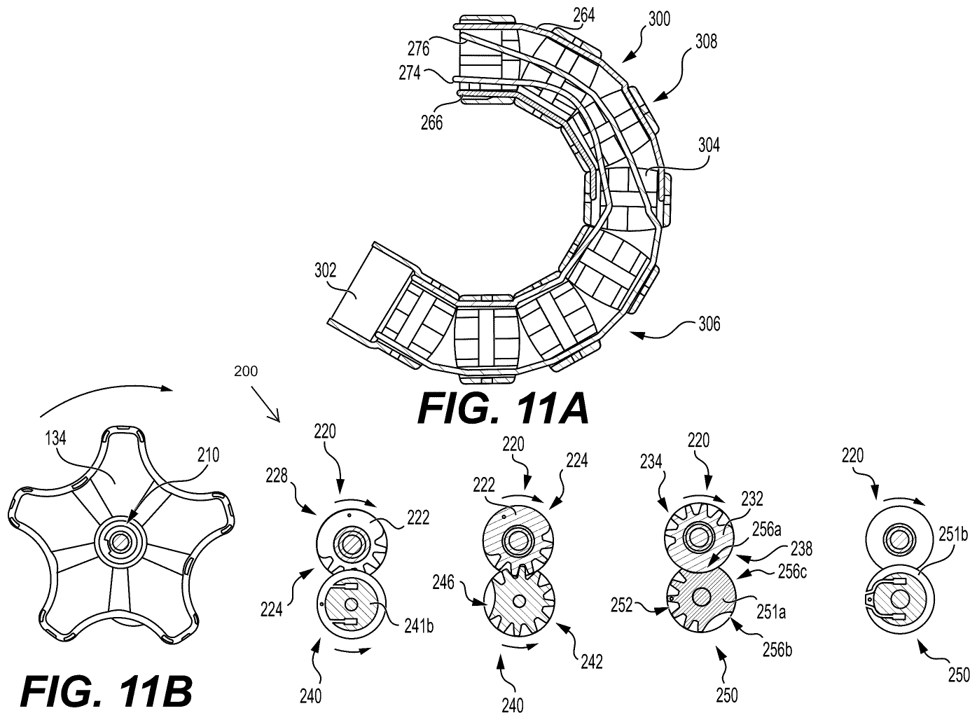

As shown in FIGS. 11A and 11B, with continued clockwise rotation of second knob 134 through the rotational range, untoothed region 238 of second portion 232 of 220 may engage (e.g., slide along) first untoothed region 256a of third gear 250. Third gear 250 may not rotate (remains stationary) during this portion of a range of motion of second knob 134. Simultaneously, toothed region 224 (a first gear first toothed region) of first portion 222 of first gear 220 may engage toothed region 242 of second gear 240, thereby rotating second gear 240 in a counter-clockwise direction. This may tension (e.g., move proximally) second articulation wire 266, causing proximal portion 308 of articulation joint 300 to bend downward to adopt a fourth articulated configuration, which may have a C-shape similar to the second articulated configuration, as shown in FIG. 11A. This may be similar to the configuration of FIG. 9A but curved downward instead of upward. Both distal portion 306 and proximal portion 308 may be curved. In any of the articulated configurations, shaft 118 may be rotated about a longitudinal axis of a proximal portion of shaft 118, such that distal tip 120 may be manipulated through a hemispherical or spherical range of motion.

In alternatives, second gear 240 may be a traditional, non-interrupted gear with teeth extending around an entire circumference of second gear 240. Alternatively, first articulation wire 264 and second articulation wire 266 may be coupled to a spool that is directly coupled to shaft 210 and does not interact with first gear 220.

In some embodiments, second gear 240 and third gear 250 may be coaxial. In some examples, second gear 240 and third gear 250 may have a same pitch of teeth 244, 254, same pitch diameters, and same diameters/widths. Alternatively, second gear 240 and third gear 250 may have different tooth pitches and pitch diameters. In aspects of such examples, first gear 220 may have different tooth pitches and diameters on first portion 222 and second portion 232. A resulting difference in gear ratios may provide for different operating torques and different articulation response rates felt by an operator. Alternatively, second gear 240 and third gear 250 may have different tooth pitches and pitch diameters, but first gear 220 may have the same pitch diameter on first portion 222 and second portion 232. In some alternatives, second gear 240 and third gear 250 may not be coaxial but may have the same pitch diameter and mate with different drive gears having different pitch diameters. The difference in gear ratios may provide for different operating torques and/or different articulation response rates felt by the operator. In further alternatives, second gear 240 and third gear 250 may have different sizes (e.g., different diameters), such that the differing diameters will provide for different operating torques and different articulation response rates felt by the operator.

Steering assembly 200 may be connected to an audible clicker or other device that creates an audible, visual, or tactile signal that indicates that the operator has exited one portion of the articulation range and entered another portion of the articulation range. Such a feedback device/system may be purely mechanical or may be electro-mechanical. For example, a controller connected to medical device 100 may display an indicator on the screen displaying the camera image.

Additionally or alternatively, first articulation wire 264 and second articulation wire 266 may be angularly offset from third articulation wire 274 and fourth articulation wire 276. For example, first articulation wire 264 may be offset by approximately 90 degrees from third articulation wire 274 and second articulation wire 266 may be offset by approximately 90 degrees from fourth articulation wire 276. For example, first articulation wire 264 and second articulation wire 266 may be coupled to left and right sides of articulation joint 300, and third articulation wire 274 and fourth articulation wire 276 may be coupled to up and down sides of articulation joint 300. A single knob 134 may be utilized to articulate in left/right and up/down directions. In such an example, third articulation wire 274 and fourth articulation wire 276 may extend only through proximal portion 308 of articulation joint 300 or may extend along an entirety of articulation joint 300, to distal link 302. Such a configuration may be useful in, for example, endoscopic retrograde cholangiopancreatography, in which steerable section 128 of shaft 118 is typically articulated fully to the right before being articulated upward.

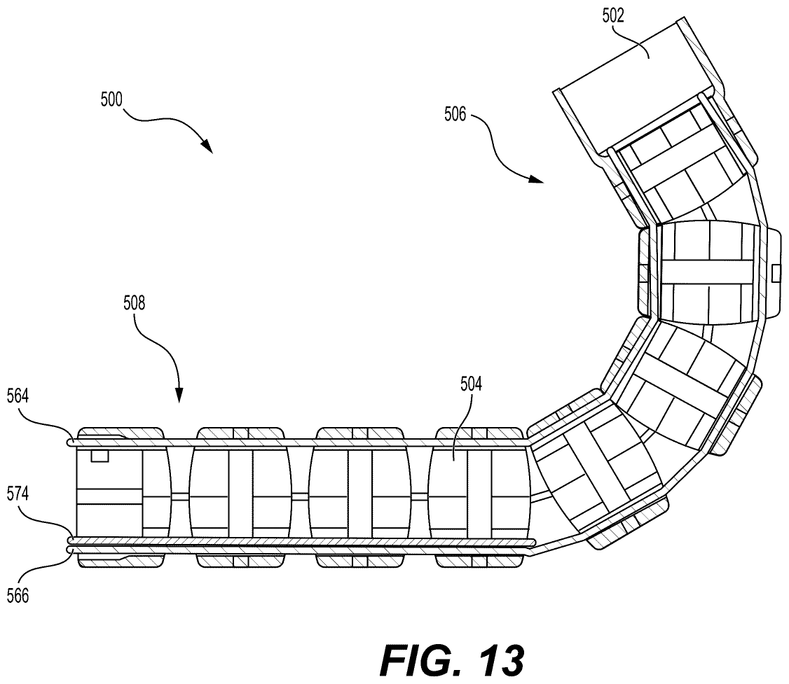

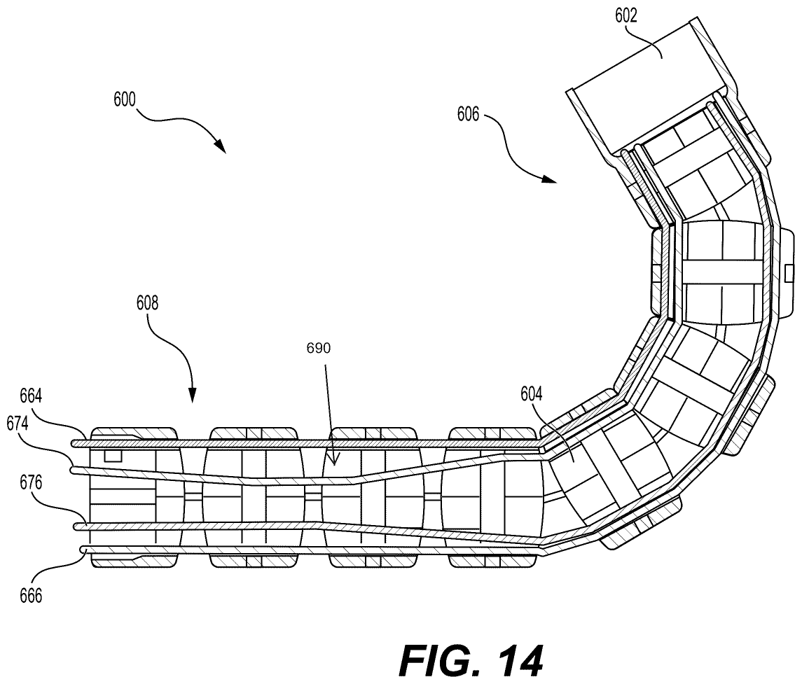

The arrangements of the portions and regions of first gear 220, second gear 240, and third gear 250 are merely exemplary, and it will be appreciated that gears with varying arrangements of toothed and untoothed regions may be utilized to provide articulation of various configurations of articulation joints. The toothed and untoothed regions may be arranged to provide for predetermined periods of time/portions of a range of rotation in which one or more gears are moving or not moving. FIGS. 13 and 14 depict exemplary alternative articulation joints that may be used with aspects of the steering assembly 200 described above.

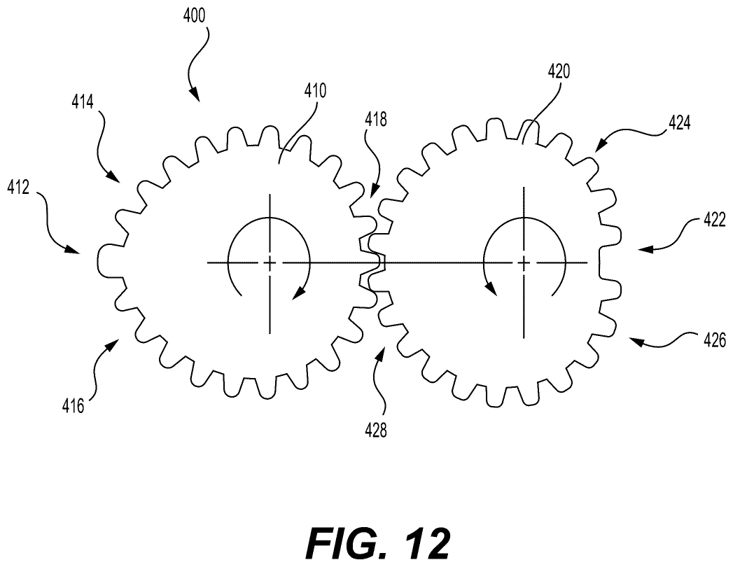

FIG. 12 depicts an assembly 400 having a first gear 410 and a second gear 420 having alternative, non-circular shapes. First gear 410 and second gear 420 may be used in place of any of first gear 220, second gear 240, and/or third gear 250. Such non-circular gears 410 and 420 may provide for different operating torques and/or different articulation response rates to be experienced by an operator. There may be a change of mechanical advantage through a range of rotation of gears 410 and 420.

First gear 410 may include an elongated end 412 and a rounded portion 418. Rounded portion 418 may have a round shape with a consistent radius. Elongated end 412 may have a larger radius than rounded portion 418. Intermediate portions 414 and 416 may taper from elongated end 412 to rounded portion 418. Second gear 420 may have a flattened end 422 and a circular portion 428. Circular portion 428 may have any of the properties of rounded portion 418 (e.g., may have a constant radius that may be the same as or different from a radius of rounded portion 418). Flattened end 422 may have a smaller radius than circular portion 428. Tapered portions 424 and 426 may connect flattened end 422 to circular portion 428.

FIG. 13 depicts an articulation joint 500. Articulation joint 500 may have any of the properties of articulation joint 300, unless otherwise specified. Whereas articulation joint 300 was described above with respect to four articulation wires 264, 266, 274, 276, articulation of articulation joint 500 in a similar manner may be controlled by only three wires. A first articulation wire 564 and a second articulation wire 566 may extend through an entire length of articulation joint 500 and may be coupled to a distal link 502. A third articulation wire 574 may be coupled to an intermediate link 504, similarly to third articulation wire 274 and fourth articulation wire 276.

As an operator turns second knob 134 in a counter-clockwise direction (e.g., an up direction), a pinion coupled to second knob 134 may rotate. The first gear may have any of the properties of first gear 220. A toothed region of the first gear may engage with a first gear that is coupled to first 564 and second articulation wire 566. First articulation wire 564 may be pulled proximally. Simultaneously, an untoothed (e.g., smooth) region of the first gear may interact with an untoothed region of a second gear that is coupled to third articulation wire 574. Third articulation wire 574 may hold or otherwise help to retain a proximal portion 508 in a straight configuration. Meanwhile, a distal portion 506 may bend into a bent configuration, for example, a J-shape, shown in FIG. 13 and similar to the shape of FIG. 8A.

Subsequently, after a predetermined amount of rotation, a toothed region of the first gear may interact with a toothed region of the third gear that is coupled to third articulation wire 574 to move third articulation wire 574 distally (de-tension third articulation wire 574). As third articulation wire 574 is moved distally, proximal portion 508 may be allowed to curve upward due to a force from first articulation wire 564, Thus, articulation joint 500 may adopt a further bent configuration, for example, a C-shape, similar to the shape of FIG. 9A.

To straighten the proximal portion 508, toothed region(s) of the first gear may interact with the second gear coupled to first articulation wire 564 and second articulation wire 566, as well as the third gear coupled to third articulation wire 574. Thus, first articulation wire 564 may be unspooled/untensioned (moved distally) while second articulation wire 566 and third articulation wire 574 are moved proximally (i.e., pulled). Because second articulation wire 566 and third articulation wire 574 overlap in proximal portion 508, proximal portion 508 may straighten first, producing the configuration (e.g., J-shape) of FIG. 13. After a predetermined amount of rotation, an untoothed region of the first gear may engage an untoothed region of the gear coupled to third articulation wire 574, such that third articulation wire 574 is retained in a predetermined position. Thereafter, further distal movement of first articulation wire 564 and further proximal movement of second articulation wire 566 may straighten articulation joint 500 to a fully straight configuration (like that of FIG. 7).

Articulation of articulation joint 500 in other directions (e.g., a down direction) may be similar to a conventional articulation joint. In some configurations of articulation joint 500, proximal portion 508 may be prevented from ever being articulated downward (a direction opposite that described above), Thus, as second articulation wire 566 is pulled proximally, articulation joint 500 may adopt an L-shape. Alternatively, pulling proximally on second articulation wire 566 may produce slack in third articulation wire 574, thereby permitting articulation joint 500 to have a downward-curving C shape (e.g., a broad C shape). Alternatively, second articulation wire 566 and third articulation wire 574 may be pulled proximally (tensioned) at the same time, which will first produce a bend in proximal portion 508 (an inverted J shape) followed by a C-shape.

FIG. 14 depicts an alternative articulation joint 600. Articulation joint 600 may have any of the properties of articulation joint 300, unless otherwise specified. Articulation joint 600 may be articulated by a first articulation wire 664, a second articulation wire 666, a third articulation wire 674, and a fourth articulation wire 676. First articulation wire 664 and second articulation wire 666 may extend through lumens of links along an entire length of articulation joint 600. In other words, first articulation wire 664 and second articulation wire 666 may be coupled to the links along an entire length of articulation joint 600 (e.g., may be coupled to all of the links). A distal end of first articulation wire 664 and second articulation wire 666 may be coupled to a distal link 602 of articulation joint 600. Combined with two other articulation wires (not shown) for articulation in a direction perpendicular to a direction of articulation controlled by wires 664, 666, 674, and 676, articulation joint 600 may be a six-wire system.

Third articulation wire 674 and fourth articulation wire 676 may be coupled only to links of articulation joint 600 that are distal to an intermediate link 604. Proximal of intermediate link 604 (in a proximal portion 608 of articulation joint 600), third articulation wire 674 and fourth articulation wire 676 may extend through a central lumen 690 of links of articulation joint 600. Proximal of intermediate link 604, third articulation wire 674 and fourth articulation wire 676 may not be coupled to articulation joint 600 and may move without moving proximal portion 608. In alternative embodiments, fourth articulation wire 676 may be omitted, and articulation joint 600 may be a five-wire system.

In operation, rotation of second knob 134 (e.g., in a counter-clockwise direction) may rotate a drive gear (e.g., a drive gear having any properties of first gear 220). The first gear may interact with a second gear that is coupled to first articulation wire 664 and second articulation wire 666 (e.g., having any of the properties of second gear 240) and a third gear that is coupled to third articulation wire 674 and fourth articulation wire 676 (e.g., having any of the properties of third gear 250). Initially, teeth of the first gear may engage teeth of the second gear and the third gear to pull proximally on both of first articulation wire 664 and third articulation wire 674 and to move second articulation wire 666 and fourth articulation wire 676 distally. Thus, a distal portion 606 of articulation joint 600 that is distal of intermediate link 604 may curve upward in a first articulated shape, such as a J-shape, as shown in FIG. 14. Proximal portion 608 may remain substantially straight.

Thereafter, after a predetermined range of motion of second knob 134, an untoothed region of the first gear may interact with an untoothed region of the third gear, such that proximal pulling on first articulation wire 664 ceases. A toothed region of the first gear may continue to engage with a toothed region of the second gear, such that first articulation wire 664 continues to be pulled proximally (and second articulation wire 666 continues to be moved distally). Articulation joint 600 may transition to a further bent configuration (e.g., a second articulated shape, such as a C-shape, similar to FIG. 9A).

When second knob 134 is subsequently rotated in an opposite direction (e.g., in a clockwise direction), the toothed region of the first gear may engage the toothed region of the second gear, to move first articulation wire 664 distally and move second articulation wire 666 proximally. Meanwhile the untoothed region of the first gear may engage with/slide along the untoothed region of the third gear, such that third articulation wire 674 and fourth articulation wire 676 do not move. This action may transition the articulation joint 600 back to the J-shape of FIG. 14.

Thereafter, as second knob 134 continues to be rotated in the opposite direction, the toothed region of the first gear may continue to engage the toothed region of the second gear, continuing to move first articulation wire 664 distally and second articulation wire 666 proximally. A toothed region (e.g., another toothed region) of the first gear may engage a toothed portion of the third gear, thereby moving third articulation wire 674 distally and fourth articulation wire 676 proximally. Such action may transition the articulation joint 600 to a straightened (relaxed) configuration, similar to FIG. 7.

In embodiments in which fourth articulation wire 676 is present, articulation in the opposite direction (e.g., downward, as second knob 134 is rotated clockwise) may be similar to the steps described above for articulating articulation joint 600 in the first (e.g., upward direction).

In embodiments in which fourth articulation wire 676 is omitted, articulation in the opposite direction (e.g., downward) may be similar to downward articulation described for articulation joint 500. A geometry of articulation joint 600 may determine a bending shape of articulation joint 600. Upon rotation of second knob 134, an untoothed region of the first gear may engage an untoothed region of the third gear to hold third articulation wire 674 in position. Simultaneously, a toothed region of the first gear may engage a toothed region of the second gear to pull second articulation wire 666 proximally and move first articulation wire 664 distally. This may cause articulation joint 600 to bend in an inverted J-shape, with a bend in proximal portion 608. Further rotation of second knob 134 may cause a toothed region of the first gear to engage a toothed region of the third gear, which may move third articulation wire 674 distally and release third articulation wire 674. Simultaneously, a toothed region of the first gear may engage a toothed region of the second gear, continuing to pull second articulation wire 666 proximally and move first articulation wire 664 distally. The articulation joint 600 may transition to a configuration in which proximal portion 608 and distal portion 606 are both curved, such as a C-shape. Continued movement of second knob 134 may transition the articulation joint 600 to a broad C-shape.

It will be appreciated that the articulation joints disclosed herein may include additional wires to those discussed above for controlling articulation in a perpendicular direction to the direction discussed above. For example, the articulation joints may include three or four wires for controlling up/down articulation and may include additional wires (two, three, or four wires) for controlling left/right articulation.

The features of the articulation joints discussed above may be combined in any suitable manner. In general, for articulation joints having four wires to control movement in a single plane (e.g., up/down movement or left/right movement), there may be various wire arrangements (which may be combined in various ways). Each of the below configurations may be controlled by three gears-a first, driving gear (e.g., a gear having any of the properties of gear 220), a second gear coupled to a first pair of wires (e.g., a gear having any of the properties of gear 240), and a third gear coupled to a second pair of wires (e.g., a gear having any of the properties of gear 250).

In a first arrangement, the second gear may be coupled to and may control articulation wires that are coupled only to a proximal portion of the articulation joint, and the third gear may be coupled to and may control articulation wires that are coupled only to a distal portion of the articulation joint. Such an arrangement may be similar to the arrangement described in FIGS. 8A-11B. In a second arrangement, the second gear may be coupled to and may control wires that are coupled to an entire length of the articulation joint. The third gear may be coupled to and may control wires that are coupled to only to a distal portion of the articulation joint. Such an arrangement may be similar to the arrangement described with respect to FIG. 14. In a third arrangement, the second gear may be coupled to and may control wires that are coupled to only a proximal portion of the articulation joint. The third gear may be coupled to and may control wires that are coupled to a full length of the articulation joint. Such an arrangement may be similar to the arrangement described with respect to the configuration described with respect to FIG. 13. In a fourth arrangement, the second gear may be coupled to and may control wires that are coupled to only a proximal portion of the articulation joint, and the third gear may be coupled to and may control wires that are coupled to only a distal portion of the articulation joint.

It will be appreciated that which gear is coupled to which pair of wires may be reversed. In such arrangements, the order in which the portions of the articulation joint are articulated may be reversed to the articulations described above. For example, a proximal portion of the articulation joint may articulate first. Alternatively, articulation joint may adopt a broadly curved “C” shape upon initial articulation (when a wire coupled to an entire length of the articulation joint is pulled) and may later adopt a “J” shape in which only the distal portion of the articulation joint is bent (when a wire coupled only to the distal portion of the articulation joint is pulled). The combinations and orders of articulation described herein are not exhaustive and the disclosure encompasses other arrangements.

It will be appreciated that, in any of the configurations described herein, one of the gears (e.g., one of gears 240 or 250) may be replaced with an untoothed pulley that is separately controlled by another shaft. Alternatively, one of gears 240 or 250 may be replaced or with a gear that is toothed around an entire perimeter of the gear and may interact with a portion of a driving gear (e.g., gear 220) that is toothed around an entire perimeter. In such a configuration, the gear with teeth around its entire perimeter may rotate whenever the driving gear is rotating.

The above exemplary articulation joints are merely exemplary and other articulation joint configurations are contemplated. In general, if there are pairs of articulation wires, then each wire of the pair will generally cross an equal number of links on each side of the articulation joint. Articulation wires may cause articulation directly or to resist articulation in a specific location from another wire. In some examples, an articulation wire may be coupled to an entire length of an articulation joint on one side of the articulation joint but only a part of the articulation joint (e.g., the proximal or distal portion of the articulation joint) on the other side of the articulation joint.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. An actuation assembly for a medical device, the actuation assembly comprising:
a first gear including:
a first gear toothed region; and
a first gear untoothed region; and
a second gear including:
a second gear toothed region;
a second gear untoothed region; and
an articulation wire coupled to the second gear;
wherein, in a first portion of a rotational range of the first gear, the first gear toothed region interacts with the second gear toothed region, such that the second gear toothed region rotates upon rotation of the first gear, and wherein, in a second portion of the rotational range, the first gear untoothed region interacts with the second gear untoothed region, such that the second gear remains stationary upon rotation of the first gear.

2. The actuation assembly of claim 1, wherein the articulation wire is a first articulation wire, and wherein the actuation assembly further comprises:
a third gear configured to interact with the first gear; and
a second articulation wire coupled to the third gear.

3. The actuation assembly of claim 2, wherein the third gear includes:
a third gear toothed region; and
a third gear untoothed region.

4. The actuation assembly of claim 3, wherein the first gear toothed region is a first gear first toothed region, wherein the first gear untoothed region is a first gear first untoothed region, wherein the articulation wire is a first articulation wire, wherein the first gear has a first portion, wherein the first portion includes the first gear first toothed region and the first gear first untoothed region, and wherein the first gear also has a second portion, wherein the second portion includes:
a first gear second toothed region; and
a first gear second untoothed region.

5. The actuation assembly of claim 4, wherein the third gear is configured to interact with the second portion of the first gear.

6. The actuation assembly of claim 4, wherein the first portion is on a first axial side of the first gear, and wherein the second portion is on a second axial side if the first gear.

7. The actuation assembly of claim 2, wherein the second gear and the third gear are coaxial.

8. The actuation assembly of claim 2, wherein, in the first portion of a rotational range of the first gear, the first gear interacts with the third gear such that the third gear remains stationary upon rotation of the first gear.

9. The actuation assembly of claim 2, wherein the first articulation wire has a distal end that is coupled to a first portion of an articulation joint, wherein the second articulation wire has a distal end that is coupled to a second portion of the articulation joint, and wherein the first portion of the articulation joint is proximal of the second portion of the articulation joint.

10. The actuation assembly of claim 9, wherein the first articulation wire and the second articulation wire are coupled to the articulation joint at a same angular position of the articulation joint.

11. The actuation assembly of claim 2, further comprising a knob, wherein rotation of the knob is configured to control movement of both the first articulation wire and the second articulation wire.

12. The actuation assembly of claim 1, wherein the second gear untoothed region is concave.

13. The actuation assembly of claim 1, wherein the second gear untoothed region is a second gear first untoothed region, and wherein the second gear further includes a second gear second untoothed region.

14. The actuation assembly of claim 1, wherein the first gear or the second gear is non-circular.

15. The actuation assembly of claim 14, wherein the third gear is non-circular.

16. An actuation assembly for a medical device, the actuation assembly comprising:
a knob;
a first member coupled to a first pair of articulation wires; and
a second member coupled to a second pair of articulation wires;
wherein, in a first portion of a rotational range of the knob, rotation of the knob causes movement of the first pair of articulation wires while the second pair of articulation wires remain stationary, and wherein, in a second portion of the rotational range of the knob, rotation of the knob causes movement of the second pair of articulation wires.

17. The actuation assembly of claim 16, wherein the knob is coupled to a first gear, wherein the first member is a second gear, and wherein the second member is a third gear.

18. The actuation assembly of claim 17, wherein the first gear includes a toothed region and an untoothed region.

19. An actuation assembly for a medical device, the actuation assembly comprising:
a first gear;
a second gear; and
a third gear;
wherein, in a first portion of a rotation range of the first gear:
teeth of the first gear engage teeth of the second gear to rotate the second gear; and
an untoothed portion of the first gear engages an untoothed portion of the third gear, such that the third gear remains stationary; and
wherein, in a second portion of a rotation range of the first gear, teeth of the first gear engage teeth of the third gear to rotate the third gear.

20. The actuation assembly of claim 19, wherein the second gear is coupled to a first articulation wire, and wherein the third gear is coupled to a second articulation wire.
